(19) United States
(12) Patent Application Publication
(10) Pub. No.: US 2005/0149996 A1
(43) Pub. Date: Jul. 7, 2005

(54) Generation of chicken cell lines from embryonic stem cells and germ cells

(76) Inventor: Alexander Baguisi, Grafton, MA (US)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 10/937,570

(22) Filed: Sep. 9, 2004

Related U.S. Application Data
(60) Provisional application No. 60/501,727, filed on Sep. 10, 2003.

(51) Int. Cl. A01K 67/027; C12P 21/04; C12N 7/00; C12N 5/06; C12N 15/86

(52) U.S. Cl. 800/6; 435/235.1; 435/456; 435/349; 435/70.21; 530/388.3

(57) ABSTRACT

The invention provides compositions of chicken cell lines and methods of production and use thereof. The cell lines are obtained from chicken germ cells or embryonic stem cells. The methods of the invention include a method of creating a chicken cell line, a method of propagating a pathogen, and a method of inhibiting tumor cell proliferation.

### RELATED APPLICATIONS

This application claims priority to provisional patent application Ser. No. 60/501,727, filed on Sep. 10, 2003, the entire contents of which are hereby incorporated by reference.

### FIELD OF THE INVENTION

The invention relates to avian cell lines and methods of creating and using same.

### BACKGROUND OF THE INVENTION

Extensive effort has been directed at developing better and more efficient methods for generating specific cell lines for commercial and research interest. The invention relates to method of generation and utilization of avian stem cell and germ cell-derived cell lines.

### SUMMARY OF THE INVENTION

The invention provides an immortalized avian germ cell and stem cell line. The cells are morphologically stable, lack pluripotency, and proliferate in the absence of growth factor supplementation. By immortalized is meant that the cells proliferate in culture beyond the Hayflick limit.

The germ cell line (GCL) is derived from primordial germ cells isolated from the gonads of day 5-6 chick embryos. The cells are characterized by a reduced amount or the loss of alkaline phosphatase activity and reduced expression of periodic acid Schiff (PAS) staining and c-Kit receptor compared to primary cultures of embryonic gonadal germ cells. The immortalized GCL (GCL-3) is deposited with the American Type Culture Collection and designated accession number American Type Culture Collection (ATCC) PTA-5436. Also within the invention is a composition containing the GCL and a virus. Preferably, the virus is selected from the group consisting of a poxvirus, an adenovirus and a retrovirus. For example, the virus is a DNA virus such as a vaccinia virus.

The invention also includes an immortalized avian stem cell and stem cell line (SCL). These cells are derived from a freshly laid fertilized egg (Stage X) and are morphologically stable, lack pluripotency, and proliferate. The cells are characterized by a reduced amount or the loss of expression of alkaline phosphatase and/or EMA-1 (embryonic mouse antigen-1) activity and reduced expression of SSEA-1 (stage specific embryonic antigen-1) and c-Kit receptor compared to primary cultures of avian embryonic cells. The immortalized SCL (SCL-1) is deposited with the ATCC and designated accession number ATCC PTA-5437. Also within the invention is a composition containing the SCL and a virus. Preferably, the virus is selected from the group consisting of a poxvirus, an adenovirus, an adeno-associated virus, and a retrovirus. For example, the virus is a DNA virus such as a vaccinia virus.

Antibodies that bind to a GCL cell and/or SCL cell such as ATCC PTA-5436 and PTA-5437 are useful to inhibit the growth of tumor cells, e.g., breast cancer, lung cancer, liver cancer cells.

The cells and cell lines are also useful to propagate replication competent viruses, replication defective viruses, and viral vectors to high titer due to their embryonic phenotype. Preferably, the viral titer at harvest is 1×10⁶ to 1×10⁷ for adenovirus and adeno-associated viruses and 1×10⁵ to 1×10⁶ for retroviruses. Production of high titer viral stock is crucial to the development of vaccines for disease prevention, GMP manufacturing of gene therapy viruses, and production of transgenic animals. The cells are contacted with a virus stock, the virus allowed to infect the cells, and the cells are maintained in culture, thereby allowing replication of the virus.

Alternatively, the establishment of an immortalized cell line can used to study the expression of embryonic antigens. The cell or cell lines are grown in vitro to high numbers, harvested, washed free of contaminating proteins, and used to immunize chickens. Cell or cell lines expressing embryonic antigens elicit an immune response against those antigens. Embryonic antigens provide useful markers for the identification of cancerous cells, and the antibodies are used to selectively kill or inhibit the growth of cancer cell bearing the embryonic antigen to which the antibody binds. Antibodies that bind to a GCL cell and/or SCL cell such as ATCC PTA-5436 or PTA-5437 have been shown useful to inhibit the growth of tumor cells, e.g., breast cancer, lung cancer, liver cancer cells. The antigen-specific polyclonal antibodies are also useful to isolate cDNA clones expressing the antigen from an SCL or GCL cDNA expression library. The antibodies are also useful to allow further characterization of the antigens as therapeutic targets or disease markers.

A method for selectively identifying embryonic antigens is carried out by using the immortalized cells (cell lines) as immunogens to immunize an avian animal such as a hen and harvesting the antibodies produced by the immunized animal to identify, characterize, and purify embryonic antigens. For example, th method is carried out by (a) immortalizing an avian germ or stem cell by blocking cell growth with mitomycin C; (b) culturing the mitomycin treated cells until they overcome the DNA synthesis block; (c) immunizing a laying hen with either live cells or a cell extract to produce antibodies against expressed embryonic antigens; and, (d) isolating antibody from eggs collected from the immunized chickens and using the antibodies to isolate, detect, or characterize the expression of the embryonic antigen, cDNA, or gene. Methods for producing an immortalized avian embryonic cell line for the propagation of viral stock are carried out by harvesting avian germ or stem cells, treating them with mitomycin C, and maintaining the cells in culture until the mitomycin C block is overcome. These methods have several advantages over earlier methods of immortalizing cells, e.g., viral immortalization or transformation with a tumor antigen. Earlier methods typically lead to expression of viral or tumor specific cell surface antigens, whereas the present method does not involve cell surface expression of exogenous antigens.

Other features and advantages of the invention will be apparent from the following description of the preferred embodiments thereof, and from the claims. The contents of references described in the specification are hereby incorporated by reference.

### BRIEF DESCRIPTION OF THE DRAWINGS

FIGS. 1a-c are photographic images demonstrating the effect of anti-SCL antibodies on SCL-1 cells. FIG. 1a. is a bright field photograph of SCL-1 cells treated with chicken primary antibody 1:500 and anti-chicken secondary FITC labeled antibody 1:2000. FIG. 1b is a merged photograph of a bright field image and a fluorescent image of SCL-1 cells treated with chicken primary antibody 1:500 and anti-chicken secondary FITC labeled antibody 1:2000. FIG. 1c is a photograph showing the immunofluorescent detection of SCL-1 cells treated with chicken primary antibody 1:500 and anti-chicken secondary FITC labeled antibody 1:2000.

FIG. 2 is a photograph showing the immunofluorescent imaging of antibody binding on GCL cells using indirect immuno-fluorescence assay.

FIG. 3 is a photograph of bright field microscopy showing apoptotic GCL cells following incubation for 7 days with 1:10 dilution of the antibody.

### DETAILED DESCRIPTION

Chicken stem cells (SCs) and germ cells (GCs) are progenitor cells known to have a broad potential for cellular differentiation into more than one type of cell lineage and have a greatly reduced incidence of immune system-mediated rejection when grafted into non-autologous hosts.

Deposit of Biological Materials

Under the terms of the Budapest Treaty on the International Recognition of the Deposit of Microorganisms for the Purpose of Patent Procedure, immortalized cell line GCL-3 (ATCC PTA-5436) and immortalized cell line SCL-1 (ATCC PTA-5437) were deposited with the ATCC, 10801 University Boulevard, Manassas, Va. 20110-2209 USA, on Sep. 4, 2003.

Applicants' assignee represents that the ATCC is a depository affording permanence of the deposit and ready accessibility thereto by the public if a patent is granted. All restrictions on the availability to the public of the material so deposited will be irrevocably removed upon the granting of a patent. The material will be available during the pendency of the patent application to one determined by the Commissioner to be entitled thereto under 37 CFR 1.14 and 35 U.S.C. 122. The deposited material will be maintained with all the care necessary to keep it viable and uncontaminated for a period of at least five years after the most recent request for the furnishing of a sample of the deposited plasmid, and in any case, for a period of at least thirty (30) years after the date of deposit or for the enforceable life of the patent, whichever period is longer. Applicant's assignee acknowledges its duty to replace the deposit should the depository be unable to furnish a sample when requested due to the condition of the deposit.

### EXAMPLE 1

### Generation of SCL and GCL Cell Lines

Isolation of the Gonads

Fertilized White Leghorn eggs from specific-pathogen free flocks were incubated for 6 days in a humidified egg incubator at 37° C. and between 85-88% relative humidity. Gonads were harvested from the developing chick embryo between 5-6 days of incubation (Hamburger stage 28-30) (Hamburger, V., and H. L. Hamilton. 1951. A series of normal stages in the development of the chick embryo. J. Morphol. 88: 49-92.). The gonads were recovered by excising the mesonephros from the genital ridge and dissecting out the gonads from the mesonephros using fine-tipped forceps under low power magnification.

The primordial germ cells (PGCs) were isolated from the gonads following standard trypsinization procedures and cultured with or without the gonadal stromal cells in tissue culture plates at 37° C. in a humidified incubator with 5% CO₂.

Purification of PGCs

PGCs were separated from the stromal cells using a standard ficoll density gradient (Yasuda, Y., A. Tajima, T. Fujimoto, and T. Kuwana. 1992. A method to obtain avian germ-line chimaeras using isolated primordial germ cells. J. Reprod. Fert. 96: 521-528). A 1.5 ml microcentrifuge tube was sequentially layered with 0.5 ml each of 16% and 7% ficoll in PGC media and overlaid with a 0.25 ml gonadal cell suspension. The gradient was centrifuged at 800×g for 30 min. The PGC-rich fraction located in the interphase between the 16% and 7% gradient was aspirated, washed, pelletted at 500×g for 5 min and re-suspended in culture media.

Isolation of Chicken Embryonic Cells (CEC)

Freshly laid, fertilized eggs (stage X; Eyal-Giladi, H., and Kochav, S., (1976) A complementary normal table and a new look at the first stages of the development of the chick. 1. General Morphology. Devel. Biol. 49:321-337) from White Leghorn (WLH) chickens were cracked open onto 100 mm Petri dishes to exposed the germinal disc. The cortically located discs were dissected and the region comprising the area pellucida was isolated using micro-dissection techniques under a stereomicroscope (Zeiss). The recovered area pellucida sections were washed in PBS (calcium and magnesium free) containing 5.6 mM D-glucose and gentamycin (50 μg/ml) and then dissociated by gentle pipetting. The cells were re-suspended in culture media and cultured in tissue culture plates at 37° C. in a humidified incubator with 5% CO_{2.}

Medium for Cell Culture

The PGCs and CECs were cultured in Dulbecco's modified Eagles medium (DMEM) with 4.5 g/L glucose (Invitrogen) supplemented with 10% FBS, 5% chicken serum, nucleosides (1 μM each nucleotide adenosine, guanosine, cytidine, uridine, thymidine), 100 U/mL Penicillin, 100 μg/mL Streptomycin and growth factors (basic Fibroblast Growth Factor, Insulin Growth Factor-1 and Stem Cell Factor at 10-100 μg/ml and murine Leukemia Inhibitory Factor at 1000-2000 units/ml) (Pain, B., M. E. Clark, M. Shen, H. Nakazawa, M. Samarut and R. J. Etches. 1996. Long-term in vitro culture and characterization of avian embryonic stem cells with multiple morphogenetic potentialities. Development 122: 2339-2348; Park, T. E and Han J. Y. 2000. Derivation and characterization of pluripotent embryonic germ cells in chicken. Molec. Reprod. Devel. 56: 475-482; Baguisi A, Masclee J, Halley D, Barry-Cedar C and Ebert K M. (2002) Avian Primordial Germ Cells: Potential for transgenesis and conservation. Theriogenology 57: 775; Baguisi A. and Ebert K M. 2002. Pharmaceuticals through Bioengineering: The Avian Alternative. In Gene Cloning and Expression Technologies. Ed. Michael Weiner and Quinn Lu, 267-279. Eaton Publishing Company, Westborough M A.)

Medium for Transformed Cells

Current medium for propagation of SCL and GCL lines is Dulbecco's modified Eagles medium (DMEM) with 4.5 g/L glucose with 10% FBS supplemented with antibiotics at 100 U/mL Penicillin and 100 μg/mL Streptomycin. Both cell lines are propagated in serum-free hybridoma medium (H-SFM).

Induction of Transformation

Secondary cultures of CECs at 75% confluence in 12-well plates were treated with Mitomycin-C (1-10 ug/ml) in culture media for 2-3 hours. The plates were washed and the cells were returned to normal culture media. Twenty-four hours after the treatment the cells were re-plated at 40-50% confluence and cultured for at least 2 weeks until cells overcome the DNA synthesis block induced by Mitomycin-C. Cell proliferation is an indication that the cells have overcome the Mitomycin-C block. The transformed cells were maintained in culture until they took over the wells from the non-transformed cells. Daughter cells in suspension were isolated and re-plated in 24-well plates. Colonies with high growth rates were selected and expanded beyond the Hayflick limit.

Isolated PGCs were seeded onto a monolayer of gonadal stromal cells and cultured until they form individual colonies and enter quiescence. Transformed CECs were introduced into the cultures and co-cultured for 2 weeks in well inserts until colonies of PGCs exited the quiescent state. Colonies that entered the proliferative phase were isolated, dispersed and reseeded for expansion. Cell lines from individual colonies that were showing morphological and growth characteristics similar to the transformed CECs were selected and expanded in culture beyond the Hayflick limit.

### EXAMPLE 2

### Characterization of SCL and GCL Cell Lines

Characterization of PGCs, CECs, and Transformed Cells

The CECs, PGCs and the transformed cells with preliminary designations of SCL lines (CEC-derived) and GCL lines (PGC-derived) respectively were characterized for the expression of known embryonic stem cell and germ cell markers. The cells were fixed in 3.7% formaldehyde and analyzed for expression of alkaline phosphatase activity and Periodic Acid-Schiff staining (Sigma Diagnostics). Detection of expression of molecular markers for SSEA-1 and 4, Oct-4, c-Kit and EMA-1 were performed using indirect immuno-fluorescence procedures. The comparative expressions of specific cellular markers are shown in Table 1.

Growth Characteristics of SCL and GCL Cells

Colonies of SCL and GCL cells derived from CECs and PGCs respectively did not vary greatly in size (15-20 microns). Cells grew as mesynchemal-type cells (fibroblast-like) on tissue culture plates at low seeding rates. Both cell derivatives show prolific and aggressive growth characteristics with a doubling time of approximately 24 hours. Both lines can induce differentiation of embryonic cells or take over in mixed cultures of somatic cells in long-term cultures. Proliferative capacity of both cell types were not inhibited at confluence and daughter cells will grow in suspension as loosely attached cell clusters. Daughter cells are propagated as suspension cultures. Short term culture in conditioned media from both SCL and GCL lines added to culture media at a ratio ranging from 1:4 up to 1:1 transiently induces proliferation of quiescent colonies of germ cells without inducing transformation.

In Vitro Differentiation Assay

To determine their pluripotent capacity in vitro, the SCL and GCL lines were removed from growth factor control and induced to spontaneously differentiate. The cell lines were found to be morphologically stable, maintain proliferative capacity and growth characteristics when cultured in media without growth factor supplementation. Both cell lines did not show pluripotent capacity indicative of stem cell plasticity expressed by non-transformed CECs and PGCs in vitro.

Soft Agar Assay

SCL-1 cells were tested for the ability to form colonies on soft agar using a standard assay. Briefly, ˜5×10³ cells suspended in 0.35% DMEM agar containing 10% fetal bovine serum (FBS) were overlaid onto a base of solidified 0.5% DMEM/10% FBS agar in a 35 mm dish. Plates were incubated at 37° C. in a 5% CO₂ environment for 10-14 days. Number and size of colonies were noted and compared with positive control culture of MES-SA cells. SCL-1 cells were found to form numerous small colonies in this assay, indicating that the line is transformed, but weakly so since colonies were smaller (by >50%) than those formed by the aggressively transformed control cells.

### EXAMPLE 3

### Producing Lentivirus in GCL-3 or SCL-1 Cells

The avian germ cell lines and stem cell lines are used as host cells for virus propagation and provide advantages to earlier methods that utilize human kidney cells, e.g., human embryonic kidney cells. The procedure described below is an example for the production of a replication deficient lentivirus in the avian cell lines GCL-3 or SCL-1 using the ViraPower™ Lentivirus kit from Invitrogen™. The method is adaptable for replication competent viruses or infectious viruses.

On Day 0 (the day prior to transfection) cells are seeded to be approximately 80% confluent by the next day (e.g. 1×10⁷ cells per 10 cm plate). Cells are plated in growth medium containing serum (i.e. DMEM+10% FBS+1% Penicillin/Streptomycin). On Day 1, the culture medium is removed and replaced with 5 mL of growth medium without antibiotics (or OptiMEM® I Medium). DNA-Lipofectamine™ 2000 complexes are prepared for each transfection sample as follows: (a) Dilute 9 μg of packaging mix and 3 μg of pLenti expression plasmid DNA in 1.5 mL of OptiMEM® I Medium without serum. Mix gently; (b) Mix Lipofectamine™ 2000 gently before use, then dilute 36 μL in 1.5 mL of OptiMEM® I Medium without serum. Mix gently and incubate for 5 minutes at room temperature; (c) Combine the diluted DNA with the diluted Lipofectamine™ 2000. Mix gently and incubate 20 minutes at room temperature. Add the complexes dropwise onto the cells. Mix gently by rocking the plate. Incubate the cells overnight at 37° C. in a CO₂ incubator.

On Day 2, the medium is removed and replaced with growth medium.

On Day 3-4, the virus-containing supernatants are harvested 48-72 hours post transfection by removing the medium and any detached cells to a 15 mL sterile, capped, conical tube. The tubes are centrifuged at 3000 rpm for 15 minuets at +4° C. to pellet cell debris The supernatant is optionally filtered through 0.45 μm filter. The virus stock (supernatant) is aliquoted into cryovials and frozen at 80° C. for storage. The titer of the virus is determined on a permissive cell line using methods known in the art.

### EXAMPLE 4

### Generation of Antibodies to the SCL Cell Lines

Production of Chicken Antibodies to SCL Cells

Laying hens were immunized by subcutaneous injection of 5×10⁶ live SCL cells in 500 ul PBS at one site and 500 μl RIBI adjuvant at a second site on days 0 and 14, followed by 5×10⁶ live SCL cells IV on day 21. One to two weeks after the third injection, eggs were collected from individual hens and the yolks processed for antibody separation and purification. Alternatively, hens are immunized with killed whole cells, cell lysates, or cell fractions emulsified with adjuvant.

A commercially available purification kit (Eggcellent™ Chicken IgY Purification kit, PN 44918) was used to isolate the antibodies from the yolk yielding IgY ranging between 80-120 mg/ml of 90% pure IgY/egg. Briefly, the egg yolk was separated from the egg white using an egg separator. The separated yolk was delipidated from 2-24 hours at refrigeration temperatures using the delipidation reagent supplied with the kit. Following delipidation, the IgY was precipitated for 1 hour and centrifuged for 15 minutes at 10 k×g in a refrigerated centrifuge. The supernatant was discarded and the IgY pellet was dissolved in PBS equal to the original egg yolk volume. The IgY concentration was determined using an A280 extinction coefficient of 1.4 equivalent to 1 mg/ml.

Antibody Detection and Bioactivity Assay

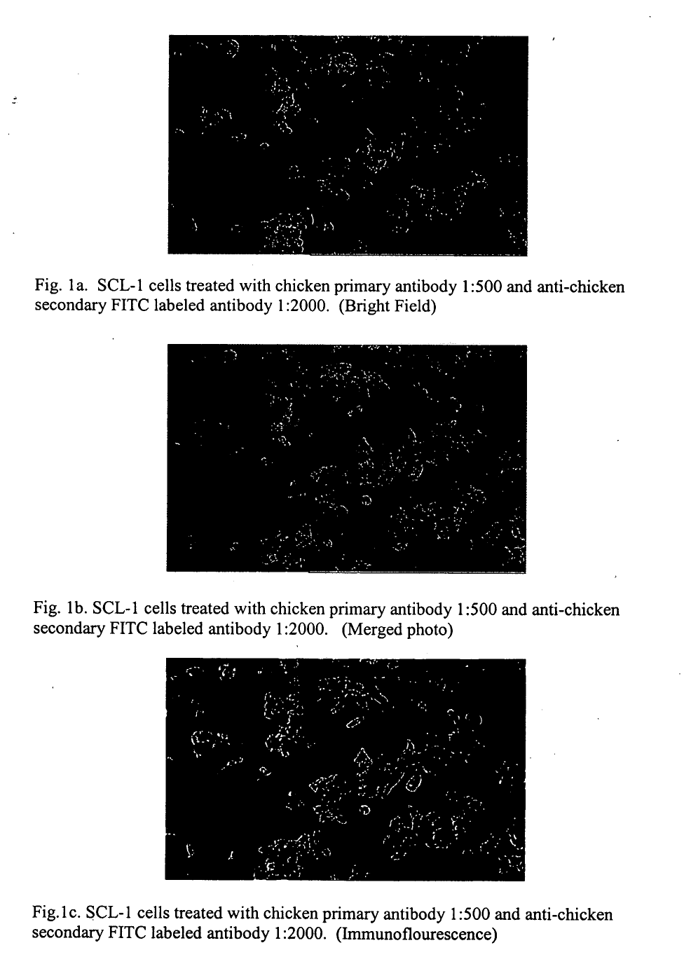

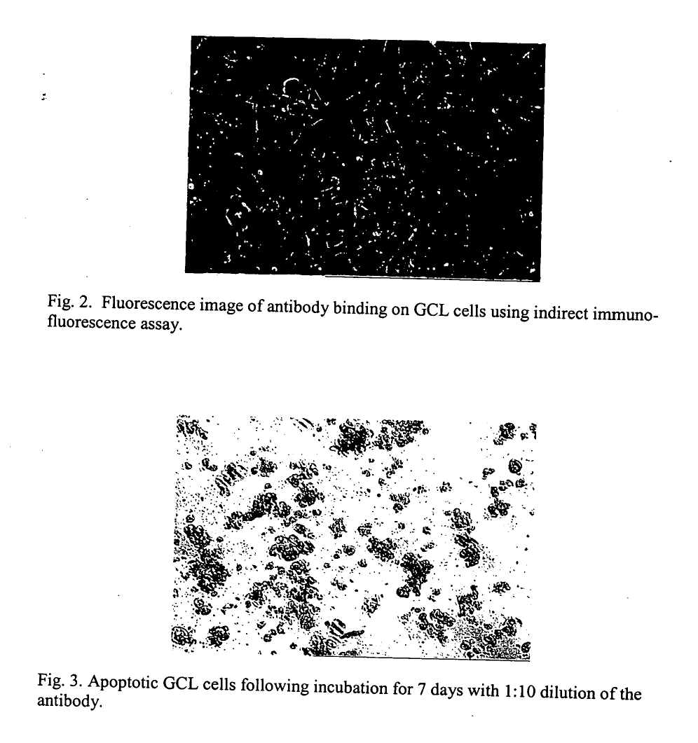

SCL cells were plated up to 50% confluence and fixed in 3.7% formalin for 15 minutes at room temperature. A crude preparation of the antibody was diluted in PBS with 1% BSA, added to the fixed cells and incubated for one hour. The cells were then washed with PBS and blocked for 1 hour in PBS with 1% BSA. After blocking, the cells were incubated with an FITC conjugated anti-chicken secondary antibody for 2 hours. Chicken antibody localization was assayed by fluorescence detection. Indirect immuno-fluorescence visualization of SCL (FIGS. 1a-c) and GCL cells (FIG. 2) indicated strong labeling of the membrane and the region of the cytosol surrounding the nuclei. Primary cultures of CECs and PGCs only showed punctuated staining of the cytoplasm. The results indicate that the antibody binds to a molecule(s) (protein) that is over-expressed in the cytosol with the active/mature form translocated in the membrane of SCL and GCL cells but not in primary CECs and PGCs from which the respective lines were derived. Western blot analysis of SCL cell fractions localized the major reactivity proteins to a low speed cellular pellet (three reactive bands of approximately 14 KDa) and a cytosol fraction (two bands between 45-60 Kda).

Bioactivity assays against SCL, GCL and DF-1 (chicken carcinoma cells and MDA cells (human breast cancer cell line) were performed in vitro. Cells at 50-75% confluence were incubated with the chicken antibody at the concentration ranging from 1:10 to 1:100 v/v for 7 days in culture media with 10% chicken serum. The cultures were assayed for proliferative capacity, occurrence of necrosis and programmed cell death at 3, 5 and 7 days of incubation. Cells were vitally stained using a triple fluorescence stain (H-33342, propidium iodide and Calcein-AM) to qualitatively assess for the presence of live, dead and apoptotic cells.

Preliminary results indicate an anti-proliferative activity of the polyclonal chicken IgY against SCL and GCL (75%), DF-1 (10%) and MDA cells (30-50%) at 1:50 dilution of the crude antibody preparation. At 1:10 dilution the antibody induced cell death to SCL and GCL cells (95-100%), DF-1 (50%) and MDA cells (75-100%) after 5-7 days of treatment. The induction of cell death was predominantly through apoptosis based on brightfield assessment (FIG. 3) and triple-stain fluorescence visualization.

### EXAMPLE 5

### Cell Line-Specific Markers in SCL-1 and GCL-3 Cell Lines

The SCL-1 and GCL-3 cell lines were tested for the expression of the stage specific embryonic antigens SSEA-1 and SSEA-4 using two different detection systems as well as for staining for alkaline phosphatase (AP), periodic acid Schiff (PAS), and other cellular markers. Ten vials of each cell line were frozen down in liquid nitrogen and tested for viability after 1 week. Viability for SCL-1 was 94%, and cells were at passage 17. Viability for GCL-3 was 96%, and cells were at passage 16. As shown in Table 1, the SCL-1 cell line is weakly positive for SSEA-1 and positive for SSEA-4. It is negative for alkaline phosphatase and positive for PAS. The GCL-3 cell line is weakly positive for both SSEA-1 and SSEA-4. It is negative for alkaline phosphatase and weakly positive for PAS. In addition, expression of EMA-1, c-Kit receptor and Oct-4 were determined. Cellular markers expressed by primary cultures of CECs and PGCs from which the SCL-1 and GCL-3 cell lines were established and are shown in Table 1.

TABLE 1
SCL and GCL-3 Cell Line Analysis

| Cell Markers | CECs | SCL-1 | PGCs | GCL-3 |
|---|---|---|---|---|
| SSEA-1 | + | +/− | − | +/− |
| SSEA-4 | ++ | ++ | − | +/− |
| Alkaline phosphatase | + | − | ++ | − |
| Periodic Acid Schiff (PAS) | | + | ++ | +/− |
| EMA-1 | + | − | − | − |
| c-Kit | +/− | − | + | +/− |
| Oct-4 | − | − | − | − |
| Population doubling period | | 20-24 hours | | 20-24 hours |

Note:
‘+’ = positive;
‘++’ = strong positive;
‘+/−‘ = weak positive;
‘−‘ = negative.

SSEA-1 and 4 and EMA-1 markers were detected using standard methods, e.g., the method described by Pain, B., M. E. Clark, M. Shen, H. Nakazawa, M. Samarut and R. J. Etches. 1996. Long-term in vitro culture and characterization of avian embryonic stem cells with multiple morphogenetic potentialities. Development 122: 2339-2348.

### Other Embodiments

Although particular embodiments have been disclosed herein in detail, this has been done by way of example for purposes of illustration only, and is not intended to be limiting with respect to the scope of the appended claims, which follow. In particular, it is contemplated by the inventors that various substitutions, alterations, and modifications may be made to the invention without departing from the spirit and scope of the invention as defined by the claims. Other aspects, advantages, and modifications are within the scope of the following claims.

1. An immortalized avian germ cell, said cell being morphologically stable and lacking pluripotency, wherein said cell proliferates in the absence of growth factor supplementation.

2. The cell of claim 1, wherein said cell is derived from a day 5-6 chick embryo.

3. The cell of claim 1, wherein said cell expresses a reduced amount of alkaline phosphatase compared to a primary embryonic gonadal cell.

4. The cell of claim 1, wherein said cell expresses a reduced amount of EMA-1 compared to a primary embryonic gonadal cell.

5. The cell of claim 1, wherein the immortalized cell line is deposited with the American Type Culture Collection and designated accession number ATCC PTA-5436.

6. A composition comprising the cell of claim 1 and a virus selected from the group consisting of an adenovirus, a poxvirus, and a retrovirus.

7. An immortalized avian stem cell, said cell being morphologically stable and lacking pluripotency, wherein said cell proliferates in the absence of growth factor supplementation.

8. The cell of claim 7, wherein said cell is derived from a freshly laid Stage X fertilized egg.

9. The cell of claim 7, wherein said cell expresses a reduced amount of alkaline phosphatase compared to a primary embryonic cell.

10. The cell of claim 7, wherein said cell expresses a reduced amount of EMA-1 compared to a primary embryonic cell.

11. The cell of claim 7, wherein the immortalized cell line is deposited with the American Type Culture Collection and designated accession number ATCC PTA-5437.

12. A composition comprising the cell of claim 7 and a virus selected from the group consisting of an adenovirus, a poxvirus, and a retrovirus.

13. A method of inhibiting proliferation of a tumor cell, comprising contacting said tumor cell with an antibody that binds to the cell of claim 5.

14. A method of inhibiting proliferation of a tumor cell, comprising contacting said tumor cell with an antibody that binds to the cell of claim 11.

15. A method of propagating a virus, comprising contacting the cell of claim 1 with said virus, wherein said virus is selected from the group consisting of a poxvirus, and adenovirus, and a retrovirus.

16. A method of propagating a virus, comprising contacting the cell of claim 7 with said virus, wherein said virus is selected from the group consisting of a poxvirus, and adenovirus, and a retrovirus.

17. A method for producing an antibody that binds to an embryonic antigen, comprising immunizing a laying hen with the cell of claim 1 or a cell extract thereof to produce an antibody in an egg, and isolating said antibody from said eggs, wherein said antibody binds to an embryonic antigen

18. A method for producing an antibody that binds to an embryonic antigen, comprising immunizing a laying hen with the cell of claim 7 or a cell extract thereof to produce an antibody in an egg, and isolating said antibody from said eggs, wherein said antibody binds to an embryonic antigen

19. A method of producing an immortalized avian embryonic cell line for the propagation of a viral stock, comprising harvesting a primary avian germ or stem cell, contacting said cell with mitomycin C, and maintaining said cell in culture until the mitomycin C block is overcome.
